(19) United States
(12) Patent Application Publication
(10) Pub. No.: US 2026/0256574 A1
(43) Pub. Date: Sep. 3, 2026

(54) POLYMERIC STRUCTURE AND METHOD OF PREPARATION THEREOF

(71) Applicant: MERIL LIFE SCIENCES PVT LTD, Vapi (IN)

(72) Inventors: PRAMOD KUMAR MINOCHA, Vapi (IN); DEVESHKUMAR MAHENDRALAL KOTHWALA, Surat (IN); AMIRHAMZAH MAHMADIQBAL SHAIKH, Valsad (IN); HAROONMOHAMMAD RASIDMOHAMMAD KHALIFA, Valsad (IN)

(73) Assignee: MERIL LIFE SCIENCES PVT LTD, Vapi, GJ (IN)

(21) Appl. No.: 19/158,038

(22) PCT Filed: Feb. 16, 2024
(86) PCT No.: PCT/IN2024/050168

§ 371 Date: Aug. 19, 2025

(30) Foreign Application Priority Data
Feb. 20, 2023 (IN) 202321011508

(51) Int. Cl. A61F 2/24

(52) U.S. Cl. CPC A61F 2/2415; A61F 2/2418; A61F 2240/004

(57) ABSTRACT

The present invention discloses a method to prepare a polymeric structure for a prosthetic heart valve. The method commences by pre-processing a polymer for a pre-defined time. The pre-processed polymer is then dissolved in at least one solvent yielding a polymer solution. Thereafter, a mold is subjected to one or more dip cycles. Each dip cycle includes inserting the mold in the polymer solution, maintaining the mold inside the polymer solution for a first hold time period, and maintaining the mold outside the polymer solution for a second hold time period. The polymer solution on the mold is then subjected to a first curing technique and a second curing technique to yield the polymeric structure. The thickness of the polymeric structure ranges from 160 microns to 200 microns. Thereafter, the polymeric structure is peeled away from the mold.

### FIELD OF INVENTION

The present invention relates to a method of preparing a polymeric structure. More specifically, the present invention relates to a method of preparing a polymeric structure for a transcatheter heart valve.

### BACKGROUND OF INVENTION

A native heart valve is made of a plurality of leaflets. The leaflets rhythmically coapt with each other to facilitate unidirectional flow of blood within the chambers of the heart. Over time, the native heart valve may deteriorate and cause life threatening conditions in an affected person. One of the major suspects for the said deterioration of the native heart valve is gradual calcium depositions (also known as calcification) over the surface of the native leaflets thereby causing damage to the native heart valve. The damaged native heart valve fails to maintain the physiological hemodynamics of a healthy heart and cause valvular insufficiency (also known as valvular regurgitation). In order to treat the damaged native heart valve, the native heart valve is replaced with a prosthetic heart valve.

Previously, the prosthetic heart valves could be implanted only via open-heart surgery requiring extracorporeal blood circulation. Given the fragility of heart patients, open heart surgery with extracorporeal blood circulation amounted to fatal consequences for most patients. In fact, many patients could not even qualify for such a sophisticated surgery thereby being denied treatment for their heart conditions.

To remedy the situation, minimally invasive procedures in the form of transcatheter aortic valve implantation (TAVI) or transcatheter heart valve replacement (TAVR) was introduced. TAVI is a percutaneous process in which the prosthetic transcatheter heart valve (THV) is implanted via a small puncture through the skin of a patient.

Generally, a THV includes an artificial valvular structure made of three leaflets supported inside an expandable frame. The leaflets of the artificial valvular structure mimic the function of healthy native leaflets thereby maintaining healthy blood flow. The leaflets of the artificial valvular structure are usually made of animal tissues (for example, bovine tissue, porcine tissue, etc.).

However, valvular structure made of an animal tissue may compromise on the hemocompatibility of the valve's material thereby providing inferior hydrodynamic performance. Further, owing to the differences in tissue degeneration and tissue lifespan of the two different species i.e., native human tissue and animal tissue of the artificial valvular structure, the risk of heart failure, stroke, or even death of patient receiving the said artificial valvular structure increases significantly. Furthermore, implantation of foreign tissue from another species always carries a risk of disease transfer from the animal to the human.

Hence, prior using animal tissue for making leaflets, it is mandatory to treat the tissue using fixative agents (usually to prevent immune response against the foreign tissue post implantation). Most common fixative agents used to fix animal tissues are aldehydes like glutaraldehyde. However, post treatment of the tissue with the fixative agent, the tissue includes an abundance of free aldehydes. These free aldehydes act as a potential calcium binding site. Thus, after implantation of the THV having the animal tissue-based leaflets, the free aldehydes induce rapid calcification of the artificial valvular structure in a short span of time thereby reducing the lifespan of the THV.

Further, the animal tissue-based leaflets include a rough surface that is prone to calcification, thrombosis and degeneration thereby limiting its durability.

In order to overcome the aforementioned disadvantages and limitations of animal tissue-based leaflets, THVs having polymeric leaflets are being explored as an alternative. Although polymeric leaflets perform better than the tissue leaflets, there still remain various challenges to be addressed that are associated with polymeric leaflets. For instance, owing to the currently employed methods by which the polymeric leaflets are made, THVs having polymeric leaflets are susceptible to high fatigue stress and degeneration of leaflets thereby resulting in complications such as poor hydrodynamic performance, mineralization, thrombosis and significant decrease in the durability of the THV.

As an example, the patent publication number US20170189175A1 discloses preparation of a polymeric heart valve by dipping the frame in a first polymeric solution and dipping the mold of a leaflet structure in a second polymeric solution. Thereafter, the polymeric frame and the polymeric valvular structure is bonded together via mechanical or chemical bonding methods such as gluing, stitching, sonic welding, mechanical fastening, etc. However, the aforesaid polymeric heart valve may not be fit for implantation due to a number of reasons. For instance, the coating of the entire frame of the prosthetic heart valve by the first polymeric solution may increase the crimped profile of the prosthetic heart valve. Furthermore, while expanding the said polymeric heart valve the coating may get fractured (or damaged) and compromise the expanding ability of the polymeric heart valve. In case the said coating gets fractured/damaged, the polymeric heart valve may give way to undesirable paravalvular leakage, especially so, in the absence of any sealing skirts. Since the entirety of the frame is coated, the polymeric heart valve may impede endothelization of the polymeric heart valve thereby negatively affecting its long-term stability. Moreover, the said polymeric heart valve may be susceptible to valvular detachment from the frame owing to improper bonding between them.

Thus, there arises a need to devise a method for preparing a polymeric structure that can overcome the issues related to conventional leaflets.

### SUMMARY OF INVENTION

The present invention relates to a method to prepare a polymeric structure for a prosthetic heart valve. The method commences by pre-processing a polymer for a pre-defined time to reduce residual moisture content of the polymer to less than 0.02% by weight of the polymer. The pre-processed polymer is then dissolved in at least one solvent yielding a polymer solution of a pre-defined concentration. The water content of the solvent is less than or equal to 0.005% (w/v). Thereafter, a mold is subjected to one or more dip cycles. Each dip cycle includes inserting the mold in the polymer solution, maintaining the mold inside the polymer solution for a first hold time period, and maintaining the mold outside the polymer solution for a second hold time period. The polymer solution on the mold is then subjected to a first curing technique at a first pre-defined temperature(s) and a first pre-defined pressure(s) for a first pre-defined time period(s). Then, the polymer solution on the mold is subjected to a second curing technique at a second pre-defined temperature(s) and a second pre-defined pressure(s) for a second pre-defined time period(s) to yield the polymeric structure. The thickness of the polymeric structure ranges from 160 microns to 200 microns. Thereafter, the polymeric structure is peeled away from the mold. The present invention further relates to a prosthetic heart valve having the aforesaid polymeric structure.

The foregoing features and other features as well as the advantages of the invention will become more apparent from the following detailed description, which proceeds with reference to the accompanying figures.

### BRIEF DESCRIPTION OF DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts an exemplary transcatheter heart valve 100 in accordance with an embodiment of the present invention.

FIG. 1a depicts an exemplary polymeric structure 103 in accordance with an embodiment of the present invention.

FIG. 2 depicts an exemplary mold 10 in accordance with an embodiment of the present invention.

FIG. 2a depicts an exemplary cross-section of the mold 10 in accordance with an embodiment of the present invention.

FIG. 2b depicts an exemplary mold 20 in accordance with an embodiment of the present invention.

FIG. 3 depicts a method 200 to prepare the polymeric structure 103 in accordance with an embodiment of the present invention.

FIG. 4 depicts a method 400 to assemble the polymeric structure 103 with the frame 101 in accordance with an embodiment of the present invention.

FIG. 4a depicts an attachment of the valvular structure 103a with the frame 101 in accordance with an embodiment of the present invention

FIG. 4b depicts an attachment of an inner sealing member 103b and an outer sealing member 103c with the frame 101 in accordance with an embodiment of the present invention.

### DETAILED DESCRIPTION OF DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of feature, structure, or characteristic described in connection with the embodiment is included exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

In accordance with the present disclosure, a method of preparing a polymeric structure is disclosed. The polymeric structure in the present invention corresponds to an assembly of a plurality of leaflets (forming a valvular structure) and/or skirt(s) to be used within a prosthetic heart valve, for example, a transcatheter heart valve (THV). The resultant THV may be used in transcatheter aortic valve implantation (TAVI) procedures or the like. Although the present invention is described with examples of transcatheter heart valves, other kinds of prosthetic valves such as surgical heart valves, venous valves, etc. are within the scope of the teachings of the present invention.

As an overview, the method of the present invention includes pre-processing of a polymer. The polymer, as selected, plays an important role in proper functioning of the prosthetic heart valve (for example, THV). Accordingly, the polymer selected in the present invention includes a biostable and biocompatible polymer that provides sufficient flexibility to the valvular structure allowing smooth and rhythmic coaptation of the leaflets during diastolic and systolic movements.

After the polymer is pre-processed, a polymeric structure is prepared by a dip coating technique. The polymeric structure formed may then be attached to a frame of the THV for subsequent use.

The polymeric structure prepared by the method of the present invention has a nearly smooth surface(s) hence is less susceptible to fatigue stress and provides better hydrodynamic performance. Further due to the nearly smooth surface(s) of the polymeric structure, the polymeric structure is resistant to calcification and degeneration thereby rendering the polymeric structure durable.

In an exemplary embodiment, the polymeric structure derived from the method of the present invention is in the form of a single partially continuous structure, i.e., the polymeric structure is formed devoid of any stitches. The single partially continuous structure of the polymeric structure makes it structurally stable, i.e., the polymeric structure of the present invention is more resistant towards wear and tear compared to valvular structures formed by stitching separate leaflets to each other. Given the lack of stitching in the polymeric structure of the present invention, the chances of paravalvular blood leakage through stitched joints is drastically reduced leading to the increase in lifespan of the THV after implantation.

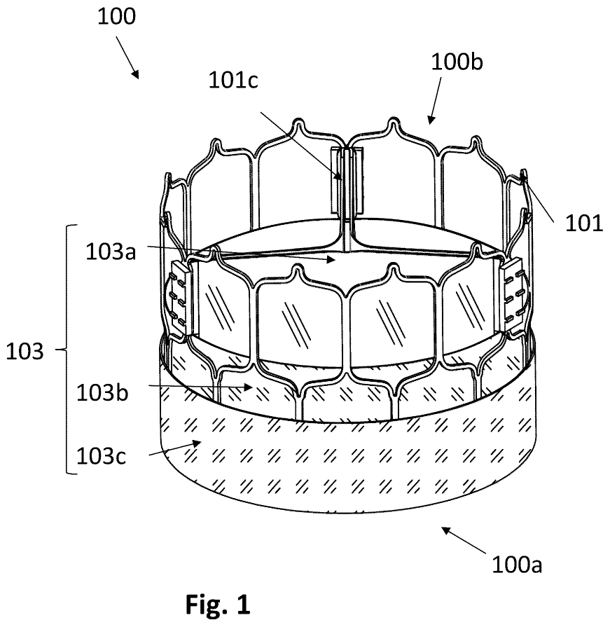

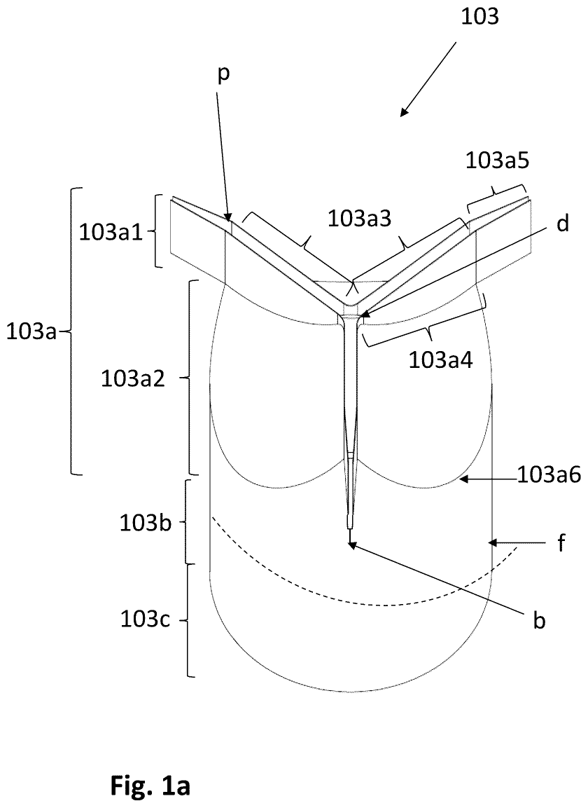

Now referring to figures, FIG. 1 depicts an exemplary embodiment of a transcatheter heart valve 100 (or THV 100). The THV 100 is a device that is implanted inside a patient's body to replace a diseased native heart valve by the functionality of a healthy native valve. The THV 100 is implanted at a target site (not shown) using a delivery catheter (not shown). The THV 100 is mounted over the delivery catheter in a radially compressed state and thereafter, radially expanded once the THV 100 is positioned at the target site. In an exemplary embodiment, the target site includes a native aortic valve of the heart.

The THV 100 may belong to one of the two categories, namely, balloon expandable or self-expandable. In case the THV 100 is a balloon expandable, once the THV 100 is positioned at the target site, a balloon of the delivery catheter is expanded to force the THV 100 to open (or radially expand) by plastic deformation. Thereafter, the balloon is deflated and the delivery catheter is withdrawn leaving the THV 100 fixed against the native heart valve and/or the arterial wall (not shown).

On the contrary, a self-expandable THV 100 is made of a ‘shape memory metal’. The self-expandable THV 100, in its radially compressed state, is held within a sheath of the delivery system during its delivery. Once the self-expandable THV 100 is positioned at the target site, the sheath is withdrawn to reveal the self-expandable THV 100 such that the self-expandable THV 100 radially expands on its own.

The THV 100 at least includes a frame 101 and a polymeric structure 103 attached to the frame 101. The THV 100 may further include an inflow end 100a and an outflow end 100b. The THV 100 may be balloon expandable or self-expandable. In an exemplary embodiment, the THV 100 is balloon expandable.

The frame 101 corresponds to an anchoring structure or a structural framework of the THV 100. The frame 101 is a tubular structure made of a bioresorbable and/or bio-compatible material(s) selected from metals or polymers. For example, the frame 101 may be made of a metallic material including but not limited to binary Nickel-Titanium alloy (nitinol), ternary Copper-Zinc-Aluminum alloy, Copper-Aluminum-Nickel alloy (or any other copper-based alloys), Cobalt Chromium alloy, etc. Alternatively, the frame 101 may be made of a polymeric material including but not limited to Poly(L-lactide) (PLLA), Poly(D, L-lactide-co-glycolide) (PLGA), Poly(ε-caprolactone) (PCL), Poly(D, L-lactide-co-glycolide) (PLGA), etc. In an exemplary embodiment, the frame 101 is made of Cobalt Chromium alloy.

The polymeric structure 103 of the THV 100 is an integral structure including one or more sections. In an exemplary embodiment, as shown in FIGS. 1 and 1a, the polymeric structure 103 includes three sections, namely, a valvular structure 103a, an inner sealing member 103b and an outer sealing member 103c. In an alternate embodiment, not shown, the polymeric structure 103 includes two sections, namely the valvular structure 103a and a sealing member (inner or outer). In yet another embodiment, not shown, the polymeric structure 103 includes one section, namely, the valvular structure 103a.

The valvular structure 103a is supported (or attached) within the frame 101 towards the outflow end 100b of the THV 100 (or the frame 101). The valvular structure 103a as per the teachings of the present invention, is made from a polymeric material, that performs better compared to a valvular structure being made of animal tissues. For example, the valvular structure 103a made from a polymeric material provides relatively better resistance towards calcification, fatigue stress, degeneration, etc. leading to superior hemodynamic performance.

As shown in FIG. 1a, the valvular structure 103a includes a top portion 103a1 and a bottom portion 103a2. The top portion 103a1 and the bottom portion 103a2 may include a pre-defined shape. In an exemplary embodiment, the top portion 103a1 may be substantially rectangular. The top portion 103a1 includes one or more free edges 103a3 and one or more attachment edges 103a4. The free edge 103a3 is disposed towards the outflow end 100b of the THV 100 with respect to the attachment edge 103a4. The free edge 103a3 may be straight or irregular. In an exemplary embodiment, as shown in FIG. 1a, the free edge 103a3 includes an undulating zig-zag pattern of alternating dips ‘d’ and peaks ‘p’. The dips ‘d’ may be center aligned with the bottom portion 103a2. And, the peaks ‘p’ may be disposed between two adjacent bottom portion 103a2. The aforesaid free edge 103a3 helps in better coaptation of the valvular structure 103a. The attachment edge 103a4 couples the top portion 103a1 to the bottom portion 103a2 (described below).

At least one attachment portion 103a5 may be provided between two adjacent attachment edge 103a4 (or the bottom portion 103a2). In an exemplary embodiment, as shown in 1a, two attachment portion 103a5 (thus, making a pair) are provided between two adjacent attachment edge 103a4 thereby providing a break ‘b’ in between the said attachment portion 103a5. In an alternate embodiment, not shown, a single continuous attachment portion 103a5 is provided between two adjacent attachment edge 103a4. The attachment portion 103a5 may be defined as a portion of the valvular structure 103a that is secured to the frame 101 of the THV 100. The attachment portion 103a5 may include a pre-defined shape including but not limited to square, rectangle, etc. In an exemplary embodiment, the attachment portion 103a5 are rectangular shaped. In an exemplary embodiment as shown in FIG. 1a, the valvular structure 103a includes three pair of attachment portion 103a5. When the valvular structure 103a is to be secured to the frame 101, the valvular structure 103a is sutured to the frame 101 with the help of the three pair of attachment portion 103a5 (described below in detail).

In an exemplary embodiment, as shown in FIG. 1a, the bottom portion 103a2 of the valvular structure 103a extends from the attachment edge 103a4. The bottom portions 103a2 may extend equidistant to each other from the attachment edge 103a4 of the top portion 103a1. The bottom portion 103a2 of the valvular structure 103a may each include a bottom edge 103a6 interrupted by attachment portion 103a5 of the top portion 103a1. The number of bottom edges 103a6 may depend upon the number of leaflets present in the valvular structure 103a. In an exemplary embodiment, the valvular structure 103a includes three U-shaped bottom edges 103a6 as shown in FIG. 1a. The bottom edges 103a6 extend away from the attachment edge 103a4 of the top portion 103a1 of the valvular structure 103a. Although the valvular structure 103a of the present invention is explained with U-shaped bottom edges 103a6, other shapes such as scalloped shaped, V-shaped, straight shaped, etc. are within the scope of the teachings of the present invention.

The valvular structure 103a may include a plurality of leaflets that mimic the function of healthy native leaflets. The number of leaflets that form the valvular structure 103a may depend upon the damaged native valve i.e., whether the damaged native valve is bicuspid/tricuspid, etc. In an exemplary embodiment, the valvular structure 103a that is intended to replace a tricuspid damaged native valve includes three leaflets.

In an exemplary embodiment, as shown in FIG. 1a, the leaflets of the valvular structure 103a form a single partially continuous structure. The single partially continuous structure of the valvular structure 103a eliminates wear and tear of the leaflets compared to when discrete leaflets are stitched together to form the valvular structure 103a.

The inner sealing member 103b may extend away from the bottom edge 103a6 of the valvular structure 103a and towards the inflow end 100a of the THV 100 (or the frame 101). The inner sealing member 103b may cover the frame 101 at least partially. The inner sealing member 103b may be disposed at an inner surface of the frame 101.

The inner sealing member 103b may be folded inside-out towards an outer surface of the frame 101 to yield a fold ‘f’. The fold ‘f’ may be disposed at the inflow end 100a of the THV 100.

The outer sealing member 103c may extend away from the fold ‘f’ over an outer surface of the frame 101. The outer sealing member 103c may cover the outer surface of the frame 101 (towards the inflow end 100a of the frame 101) at least partially. The inner sealing member 103b along with outer sealing member 103c prevent paravalvular blood leakage.

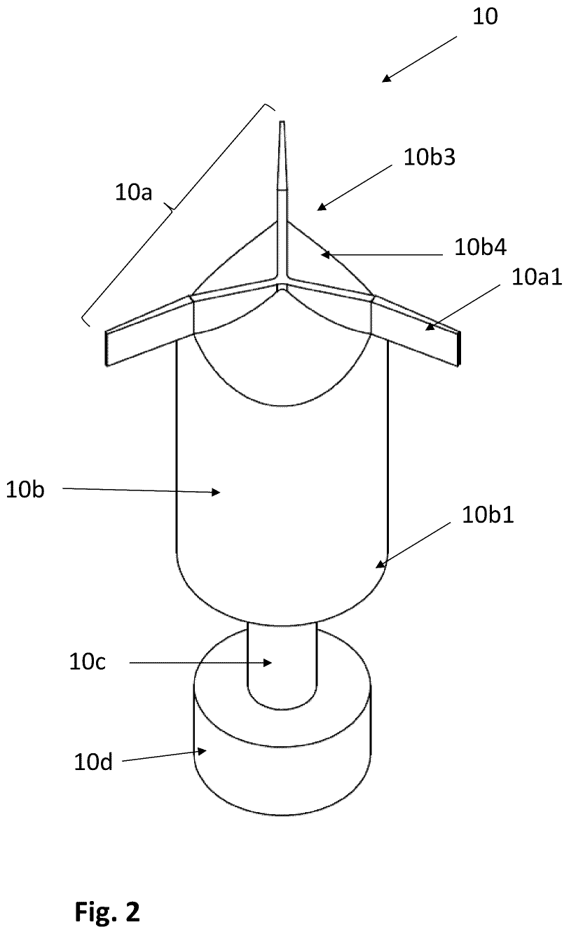

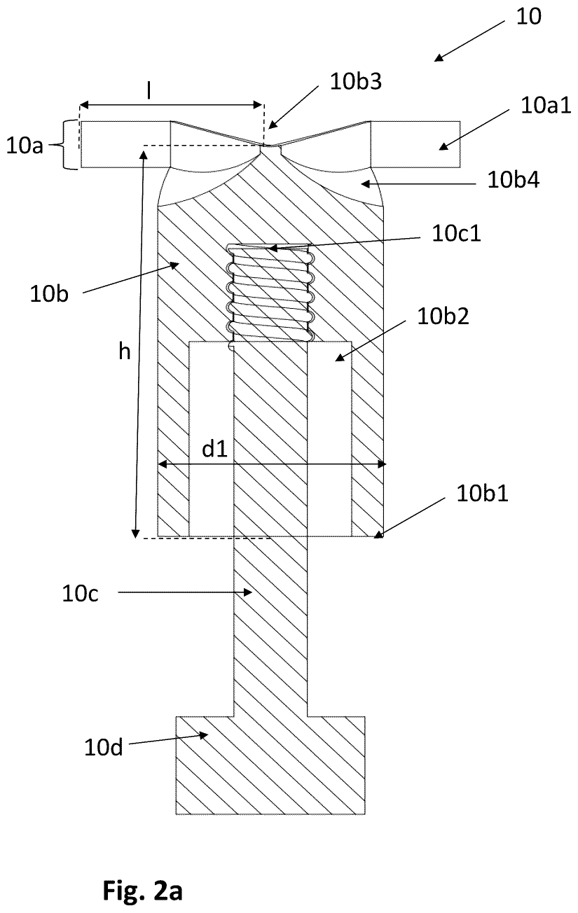

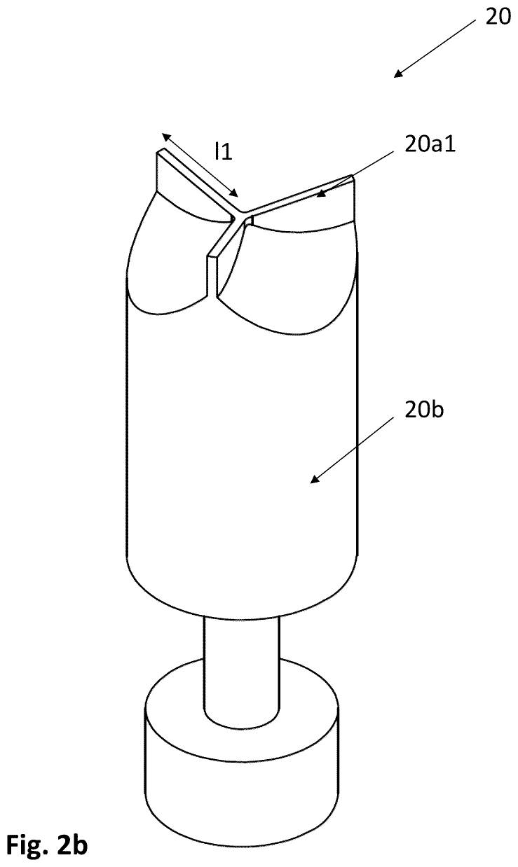

The polymeric structure 103 of the present invention may be shaped using a pre-defined device. In an exemplary embodiment, the polymeric structure 103 is made with the help of a mold 10 (as shown in FIGS. 2 and 2a). The mold 10 may be made of a predefined material including but not limited to medical grade stainless steel, glass, Teflon etc. In an exemplary embodiment, the mold 10 is made of stainless steel.

The mold 10 may include a predefined structure having one or more portions. In an exemplary embodiment, as shown in FIGS. 2 and 2a, the mold 10 includes four portions namely, a first portion 10a, a second portion 10b, a third portion 10c and a fourth portion 10d. The said portions of the mold 10 may be removably or permanently coupled to each other.

The first portion 10a may include a predefined structure including a plurality of wings 10a1. In an exemplary embodiment, the wings 10a1 is separated from each other by equal angles. The wings 10a1 provide shape to the top portion 103a1 of the valvular structure 103a. In an exemplary embodiment, as shown in FIG. 2a, the first portion 10a includes three wings 10a1. The wings 10a1 may be removably or permanently attached to each other by an attachment means including but not limited to welding, machining, injection molding, etc. In an exemplary embodiment, the wings 10a1 are manufactured as an integral unit by machining.

The wings 10a1 may have a predefined length (l). The wings 10a1 may have a length (l) either equal to or more than a radius of the second portion 10b. In an exemplary embodiment, as shown in FIGS. 2 and 2a, the length (l) of the wings 10a1 is more than the radius of the second portion 10b.

FIG. 2b depicts an alternate embodiment of a mold 20. The mold 20 is same as mold 10 except for a length (l1) of the wings 20a1. As shown in FIG. 2b, the length (l1) of the wings 20a1 is equal to a radius of a second portion 20b.

Going back to FIGS. 2 and 2a, the first portion 10a and the second portion 10b may be coupled to each other by, without limitation, mechanical fastening, gluing, welding, machining, injection molding, etc. In an exemplary embodiment, the first portion 10a and the second portion 10b are manufactured as an integral structure via machining.

The second portion 10b may have a predefined shape including but not limited to rod, cylindrical, barrel, etc. In an exemplary embodiment, the second portion 10b is cylindrically shaped. The second portion 10b may further help to shape the inner sealing member 103b and the outer sealing member 103c of the polymeric structure 103. As shown in FIG. 2a, the second portion 10b may include a pre-defined diameter (d1) and height (h) depending upon a size of the THV 100. In an exemplary embodiment, the diameter (d1) and height (h) of the second portion 10b is 23 mm and 38 mm respectively. The radius of the second section is defined as half of the diameter.

The second portion 10b includes a first end 10b3. The first end 10b3 of the second portion 10b may be coupled to the first portion 10a. The second portion 10b may include a pre-defined pattern 10b4 spread at least partially across the height of the second portion 10b. The pre-defined pattern 10b4 may be disposed adjacent to the first end 10b3 of the second portion 10b. In an exemplary embodiment, as shown in FIGS. 2 and 2a, the pre-defined pattern 10b4 of the second portion 10b includes three groove like cut-outs. The pre-defined pattern 10b4 provides shape to the bottom portion 103a2 of the valvular structure 103a.

The second portion 10b and the third portion 10c may be removably coupled via without limitation screw fit, adhesive, etc. In an exemplary embodiment as shown in FIG. 2a, the third portion 10c is screwed within the fourth portion 10d. The second portion 10b may include a second end 10b1. The second end 10b1 may include a hollow cavity 10b2 provided with a plurality of threads at least partially spread across a depth of the hollow cavity 10b2.

The third portion 10c may include a third end 10c1. The third end 10c1 of the third portion 10c may include a plurality of threads corresponding to the threads provided inside the hollow cavity 10b2 of the second portion 10b. The said plurality of threads may be disposed at least partially across a length of the third portion 10c. The threads of the third portion 10c may be screwed within the threads of the second portion 10b to couple the section portion 10b to the third portion 10c.

In an alternate embodiment, the second portion 10b is welded to the third portion 10c.

The third portion 10c may include a predefined shape including but not limited to rod shape, cylindrical shape, triangular rod shape, square rod shape, hexagonal rod shape etc. In an exemplary embodiment, as shown in FIGS. 2 and 2a, the third portion 10c is rod shaped.

The third portion 10c has a predefined diameter and a predefined height. The pre-defined height of the third portion 10c may be more than or equal to the height ‘h’ of the second portion 10b. In an exemplary embodiment, the diameter and height of the third portion 10c is 7 mm and 48 mm respectively.

The mold 10 may be operated by interacting with the fourth portion 10d of the mold 10. The fourth portion 10d may include a predefined shape including but not limited to cylindrical shaped, triangular shaped, square shaped, hexagonal shaped, rod shaped, etc. In an exemplary embodiment, the fourth portion 10d is cylindrical shaped. The fourth portion 10d may be integrally or removably coupled to the third portion 10c of the mold 10.

Further, the fourth portion 10d may include a predefined diameter and a predefined height. The predefined diameter and the predefined height of the fourth portion 10d may be less than or equal to that of the second portion 10b. In an exemplary embodiment, the diameter and height of the fourth portion 10d is 18 mm and 10 mm respectively.

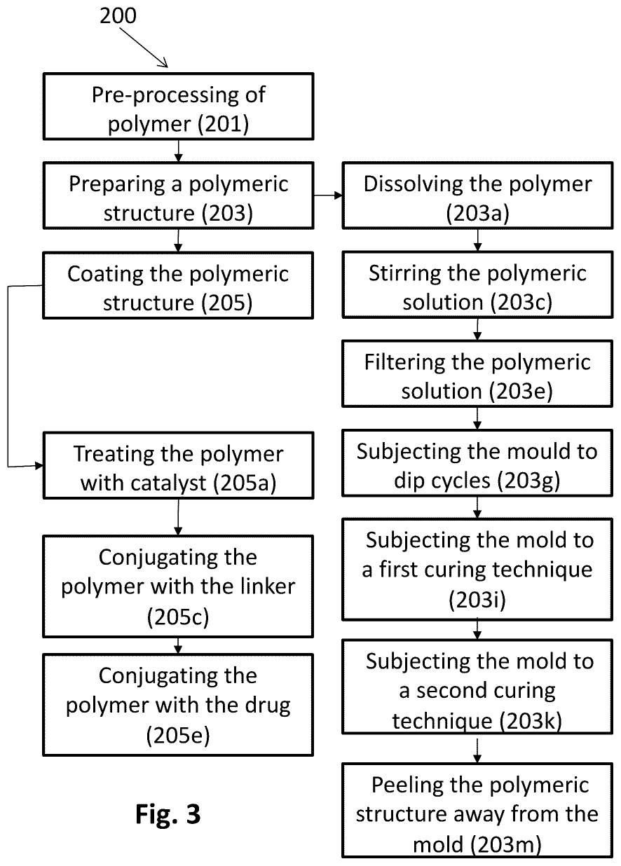

FIG. 3 depicts a method 200 for preparing the polymeric structure 103 for a prosthetic heart valve (for example, THV 100) using the mold 10 (or mold 20). Due to lack of any stitching in the single partially continuous structure of the polymeric structure 103 formed from the method 200 of the present invention, the chances of paravalvular blood leakage are reduced leading to increased life of the THV 100 after implantation.

Although the method 200 is described to prepare the single partially continuous structure of the polymeric structure 103, the preparation of polymeric structure with discrete leaflets (not shown), valvular structure (not shown), inner sealing member (not shown) and/or outer sealing member (not shown) is within the scope of the teachings of the present invention.

The polymeric structure 103 may be made of a biostable and/or biocompatible polymer. The biostable and biocompatible polymer may be selected from a group of polyurethane or block co-polymers thereof including but not limited to polyurethane, poly ether urethane polymers, polycarbonate urethane polymer, etc. The use of biostable and/or biocompatible polymers provide sufficient flexibility to the polymeric structure 103 thereby allowing easy rhythmic movement of the leaflets of the valvular structure 103a during diastolic and systolic cycles of the heart. In an exemplary embodiment, the polymer used to form the polymeric structure 103 is polycarbonate urethane block co-polymer. Polycarbonate urethane polymer provides resistance to calcification, thrombogenesis and degradation thereby increasing the operating life of the THV 100 to more than 15 years.

The method 200 commences at step 201 in which the polymer is pre-processed. The pre-processing of the polymer includes drying the polymer for a pre-defined time to remove any residual moisture (or dehydrate). The pre-defined time may be optimized for minimal to none moisture content in the polymer. The pre-defined time may range from 12 hours to 24 hours. The polymer may be in the form of pellets, powder, granules, etc. In an exemplary embodiment, the polymer is in the form of pellets. The polymer can be dried using a pre-defined method including but not limited to a vacuum desiccator, a vacuum oven, a desiccant type de-humidifying dryer, a hot air oven, etc. In an embodiment, the polymer pellets are dried in a vacuum desiccator for 24 hours at a temperature of 37±5° C.

It should be noted that the polymer (in solid and/or un-dissolved state) as available to be used for pre-processing may be hygroscopic in nature, i.e., the polymer may absorb moisture from the surrounding environment. Any residual moisture in the polymer leads to formation of bubbles and haziness during subsequent steps of the method 200. The formation of bubbles and haziness may directly affect the mechanical properties (such as decrease in elongation and tensile strength) of the polymeric structure 103 thereby reducing the durability of the polymeric structure 103. Thus, pre-processing the polymer by drying the polymer helps to reduce the moisture thereby avoiding formation of bubbles and haziness in subsequent steps of the method 200 (and the polymeric structure 103 obtained at the end). In an exemplary embodiment, the moisture content of the polymer at the end of step 201 is reduced to less than 0.02% by weight of the polymer.

At step 203, the polymeric structure 103 is formed from the pre-processed polymer obtained at step 201. In an exemplary embodiment, the polymeric structure 103 is formed by a dip coating technique. The preparation of the polymeric structure 103 by the dip coating technique enables easy and fine control over a thickness of the polymeric structure 103 in an economic way.

The dip coating technique to obtain the polymeric structure 103 is executed in a controlled environment such that the mechanical properties (such as elongation, tensile strength, etc.) of the polymeric structure 103 (and/or the polymer) is preserved. The controlled environment corresponds to an enclosed space with pre-defined humidity and temperature. The humidity may range from 25% to 50% and the temperature may range from 18° C. to 28° C. In an exemplary embodiment, the humidity and temperature of the controlled environment is maintained at 33±1% and 22±1° C. respectively. The aforesaid humidity and temperature of the controlled environment enables preparation of clear and transparent polymeric structure 103 without any haziness.

At step 203a, the dip coating technique includes dissolving the (pre-processed) polymer in a suitable solvent, for example a polar organic solvent to obtain a polymer solution. The polymer may be dissolved in the polar organic solvent at a pre-defined concentration ranging from 3% to 11% (w/v). The polar organic solvent may be at least one of dimethyl acetamide (DMAc), tetrahydrofuran (THF), dimethyl sulfoxide (DMSO), and the like. Similar to the dehydrated polymer, anhydrous solvents are preferred for preparation of the polymeric structure 103. In an exemplary embodiment, the solvent having water content less than or equal to 0.005% (w/v) is preferred for dissolving the polymer.

Further, the polymer may be dissolved in a single solvent or in a blend of two or more solvents in a pre-defined ratio in order to preserve the mechanical properties (such as elongation, tensile strength, etc.) of the polymer in the obtained polymeric structure 103. In an exemplary embodiment, the polymer is dissolved in a blend of two solvents having different boiling points. For example, a blend of DMAc (having high boiling point) and THF (having low boiling point) is used. The aforesaid blend of two solvents facilitates evaporation of the solvents at an even pace (not very slow and no very fast) during drying (described below) thereby creating a stable and homogenous polymeric structure 103 with good morphological and mechanical properties.

In an exemplary embodiment, 6% (w/v) of the polymer is dissolved in a blend of anhydrous DMAc and THF. The anhydrous DMAc and THE may be blended in a predefined ratio of 90:10, 80:20, 70:30, 60:40, 50:50, 40:60, 30:70, 20:80, or 10:90. In an exemplary embodiment, the anhydrous DMAc and THE is blended in the ratio of 10:90. The aforesaid ratio helps in the formation of the polymeric structure 103 having desired thickness and devoid of any bubbles and haziness.

At step 203c, after the polymer is dissolved in the solvent, the obtained polymer solution may be stirred to make it homogenous. The polymer solution is stirred using a predefined technique. The predefined technique may include, without limitation stirring motors, magnetic stirrers, overhead geared stirrer, etc. In an exemplary embodiment, the polymer solution is stirred using an overhead geared stirrer. The stirring of the polymer solution is done at the room temperature at a predefined rotational speed for a pre-defined time. In an exemplary embodiment, the solution is stirred at 1500 rotations per minute (rpm) for 7-8 hours. Stirring the polymer solution at the aforesaid rotational speed and pre-defined time eliminates any possibility of formation of clumps or residues and allows the polymer to be completely dissolved in the solvent thereby rendering the polymer solution homogenous.

At step 203e, optionally or additionally, the stirred polymer solution may be filtered through a mesh filter to remove any particulate matter (if present or accidentally introduced).

At step 203g, the mold 10 may be dipped one or more times into the polymer solution obtained at step 203c (or 203e). In other words, the mold 10 may be subjected to one or more dip cycles such that the first portion 10a and the second portion 10b is coated at least partially with the polymer solution. In an exemplary embodiment, the polymer solution contacts the first portion 10a of the mold 10 prior to the second portion 10b of the mold 10 in the dip cycle(s). In an exemplary embodiment, the third portion 10c and the fourth portion 10d do not contact the polymer solution during the dip cycle(s).

Each dip cycle may correspond to at least partially inserting (and maintaining) the mold 10 into the polymer solution up to a predefined height of the mold 10 (or the second portion 10b of the mold 10) for a first hold time period(s) and withdrawing (and maintaining) the mold 10 from the polymer solution at least partially thereafter. The mold 10 may be kept out of the polymer solution for a second hold time period(s) between two consecutive dip cycles. The first hold time period and the second hold period time may range from 1 second to 10 second. The pre-defined height of the mold 10 being dipped into the polymer solution may depend upon a height of the inner sealing member 103b and/or outer sealing member 103c as required. In an exemplary embodiment, the pre-defined height of the mold 10 being dipped within the polymer solution is equal to 30 mm. In an exemplary embodiment, the mold 10 is inverted and then dipped into the polymer solution such that the first portion 10a comes in contact with the polymer solution first followed by the second portion 10b.

The concentration of the polymer solution and/or the number of dip cycles may influence the thickness of the polymeric structure 103. For instance, a dilute polymer solution may require more number of dip cycles relative to a concentrated polymer solution to achieve the same thickness of the polymeric structure 103. The thickness of the polymeric structure 103 may be in the range of 160 to 200 microns. In an exemplary embodiment, the thickness of the polymeric structure 103 is 180±10 micron. The polymeric structure 103 having the aforesaid thickness provides large effective orifice area (EOA) for better hydrodynamic performance and enables low-profile crimping of the THV 100 over the delivery device.

In an exemplary embodiment, the polymeric structure 103 with a thickness of 180 microns is obtained by dipping the mold 10 for 10 dip cycles in a 6% (w/v) polymer solution prepared in a blend of DMAc and THF (blended in a ratio of 10:90). The said dip cycles included the first hold time period of 5 seconds and the second hold time period of 10 seconds.

The dip cycles of the mold 10 may be executed manually or by a (semi) automatic dipping apparatus. The (semi) automatic dipping apparatus may execute the dip cycles thereby requiring less time and manpower for making the polymeric structure 103 with high precision and accuracy. In an exemplary embodiment, the dip cycles of the mold 10 is carried out physically by a person holding the mold 10 via the fourth portion 10d.

At step 203i, the mold 10 (at least partially covered in the polymer solution) may be subjected to a first curing technique at a first pre-defined temperature(s) and a first pre-defined pressure(s) for a first pre-defined time period(s). The first pre-defined temperature may range from 30° C. to 40° C. The first pre-defined pressure may range from 650 mmHg to 700 mmHg. The first pre-defined time period may range from 5 minutes to 15 minutes. The first curing technique may be conducted inside, without limitation hot air oven, vacuum oven, etc. In an exemplary embodiment, the mold 10 is subjected to the first curing technique by placing the mold 10 inside a vacuum oven maintained at 37° C. temperature and 650 mmHg pressure for 10 minutes. The first curing technique helps to produce a transparent polymeric structure 103 without any bubbles and haziness.

At step 203k, the mold 10 may be subjected to a second curing technique at a second pre-defined temperature(s) and a second pre-defined pressure(s) for a second pre-defined time period(s). The second pre-defined temperature may range from 40° C. to 60° C. The second pre-defined pressure may range from 650 mmHg to 700 mmHg. The second pre-defined time period may range from 3 hours to 4 hours. The second curing technique may be conducted inside, without limitation hot air oven, vacuum oven, etc. In an exemplary embodiment, the mold 10 is subjected to the second curing technique by placing the mold 10 inside a vacuum oven maintained at 60° C. temperature and 650 mmHg pressure for 4 hours. The second curing technique ensures complete evaporation of the solvent from the polymer solution covered mold 10 to yield the polymeric structure 103.

At step 203m, the polymeric structure 103 may be peeled away from the mold 10. In an exemplary embodiment, the polymeric structure 103 is peeled away from the mold 10 with the help of forceps under running purified water. Running purified water helps in easy removal of the polymeric structure 103 away from the mold 10 without any damage to the polymeric structure 103.

Optionally or additionally, at step 205, a pre-defined coating is disposed over the polymeric structure 103 as obtained in the step 203. The pre-defined coating of the polymeric structure 103 helps to repel any particulate matter present in the blood thereby minimizing risk of thrombosis and valve failure.

In an exemplary embodiment, the polymeric structure 103 is coated only if the receiving patient (person with a diseased native heart valve) is not dependent upon any blood thinners or anti-coagulant agents in their daily life.

In an alternate embodiment, the polymeric structure 103 without any coating is prescribed if the receiving patient is dependent on blood thinners or anti coagulating agents in their daily life or patients who have high thrombin time where the blood clotting proteins do not produce thrombin enzyme leading to inhibition of fibrin formation from fibrinogen and ultimately failing to produce fibrin clots.

The pre-defined coating may be coated and/or conjugated over the polymeric structure 103 with agents including but not limited to, anti-thrombotics, thrombolytics, antiproliferatives, anti-inflammatories, antimitotic, antimicrobial agents, etc.

In an exemplary embodiment, the pre-defined coating includes anti-thrombotic agents. The anti-thrombotic agents may include, without limitation anti-platelet or anti-coagulants. In an exemplary embodiment, an anti-coagulant agent such as heparin is used to coat the polymeric structure 103. Heparin is a glycosaminoglycan which prevents thrombosis over a surface of the polymeric structure 103 that actively come into contact with blood.

In an alternate embodiment, the pre-defined coating is a conjugation of anti-coagulant with polycarbonate urethane (PCU). The said conjugation may be done using a pre-defined technique including, without limitation covalent linkage, physical adsorption, ionic bonding and photo-grafting.

In an exemplary embodiment, heparin is conjugated to the polymeric structure 103 by a three-step covalent bonding method. The first step 205a (depicted below), PCU polymer's surface may be treated with a catalyst such as di-n-butyl tin dilaurate (DBTL) to link the isocyanate functional group i.e., hexamethylene diisocyanate (HDI) to generate HDI linked PCU. In order to prevent the absorption of isocyanate into the PCU polymer's surface, poor solvents such as toluene, xylene, and the like may be used for the first step.

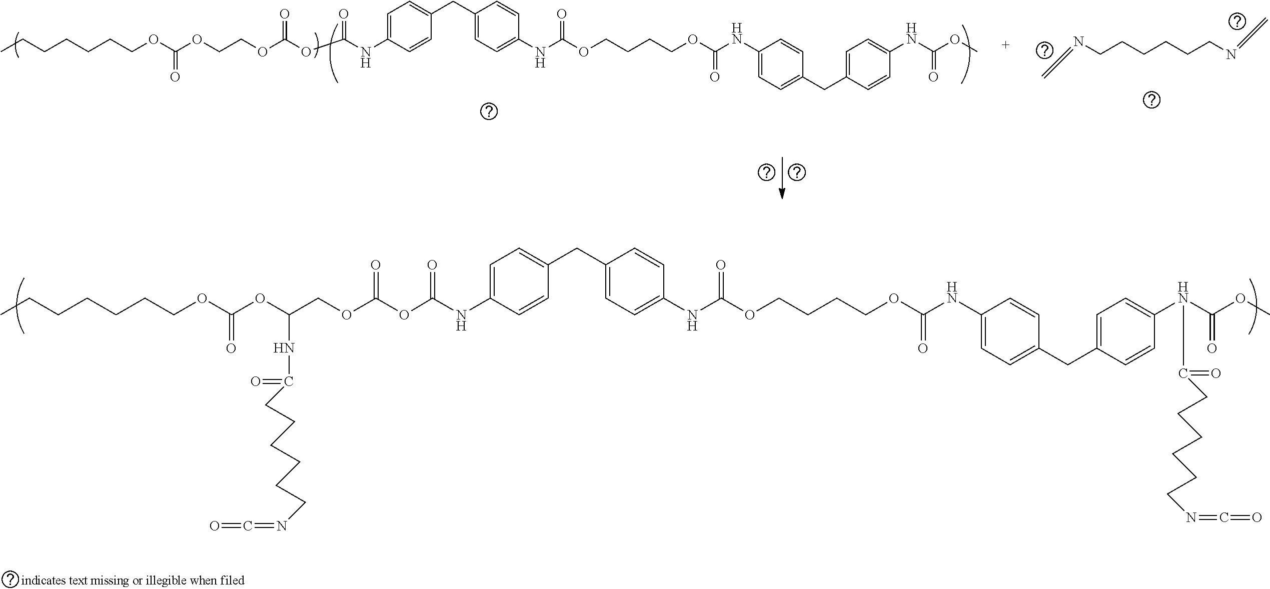

The second step 205c (depicted below) includes conjugating the HDI linked PCU polymer's surface to α, ω-diamino polyethylene glycol (APEG) to produce the APEG conjugated PCU polymer's surface. The APEG acts as a spacer (or linker) between the polymeric structure 103 and the pre-defined coating (i.e., the heparin) which improves the ability of heparin to form a direct, adherent, and strong bonding with the polymer's surface while decreasing the adsorption of non-specific proteins.

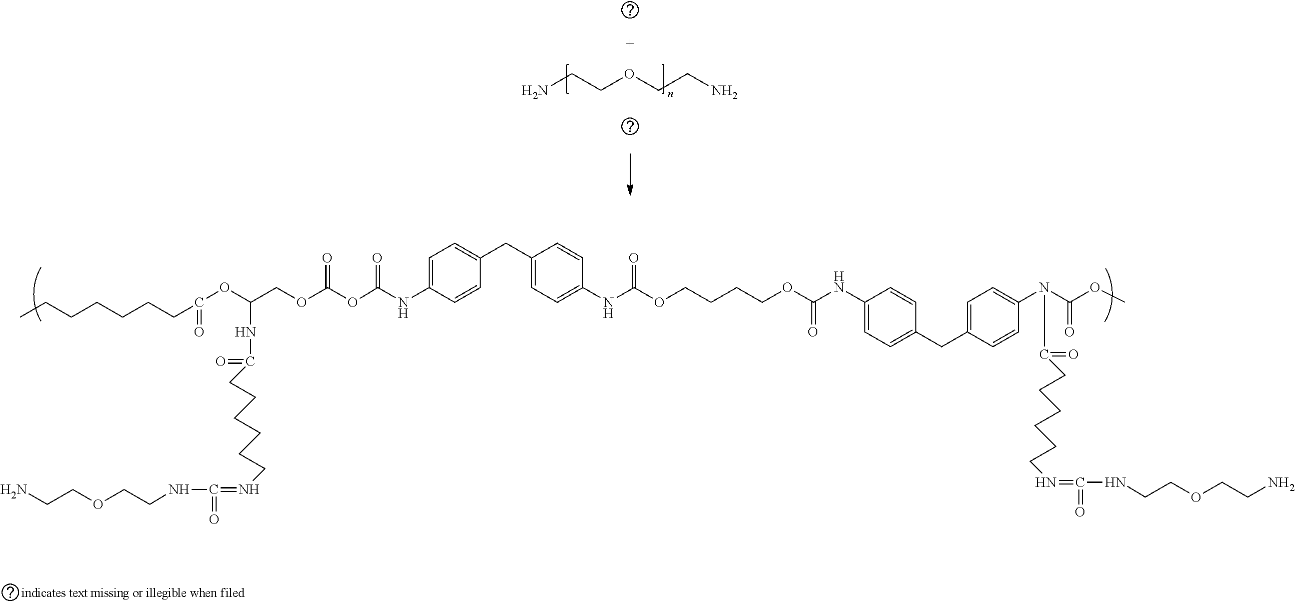

At the third step 205e (depicted below), the APEG conjugated PCU polymer surface is then covalently conjugated to heparin (or any other drug as required) in the presence of 1-ethyl-3-(3-dimethylaminopropyl) carbodiimide (EDAC) and N-hydroxysuccinimide (NHS) to form heparin linked PCU polymer's surface.

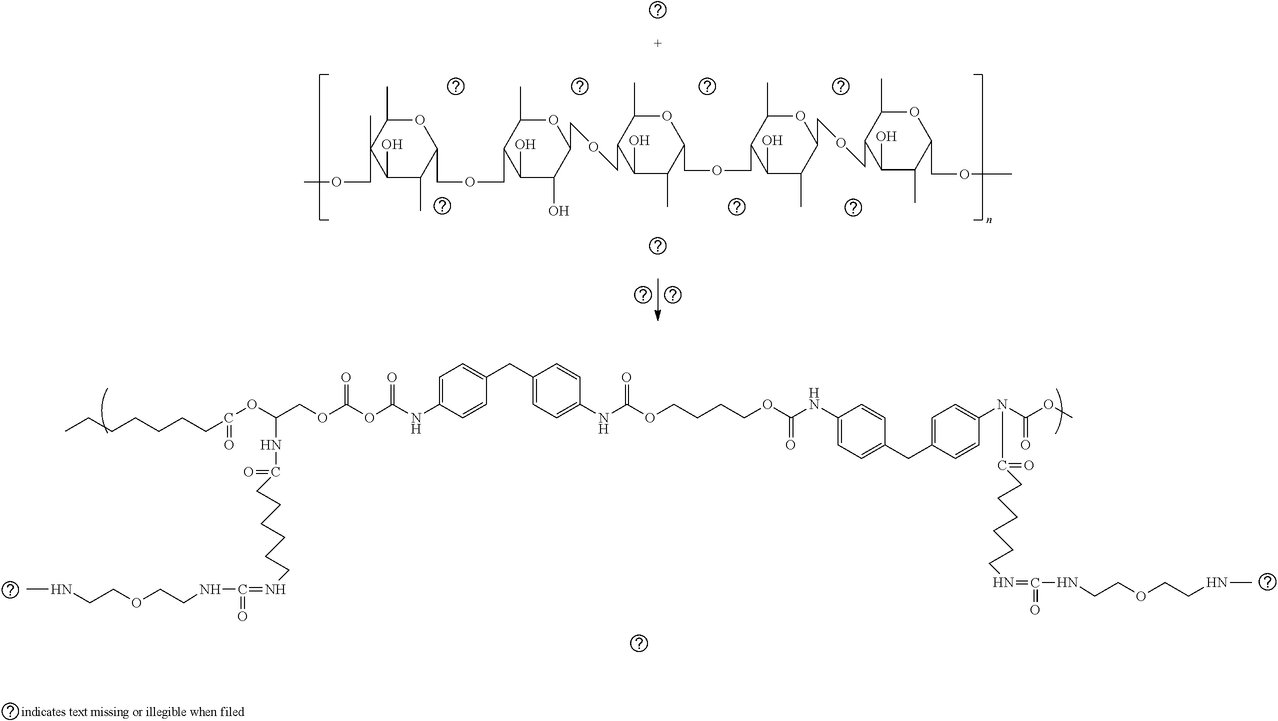

The polymeric structure 103 may be mounted within the frame 101 of the prosthetic heart valve (for example, THV 100) before it is implanted at the target site. It should be noted that though the present invention is explained via examples of transcatheter heart valves, the polymeric structure 103 may be mounted within any prosthetic valves.

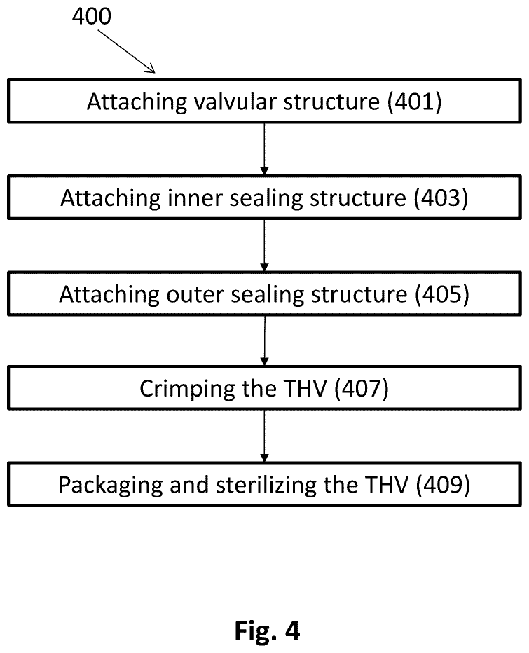

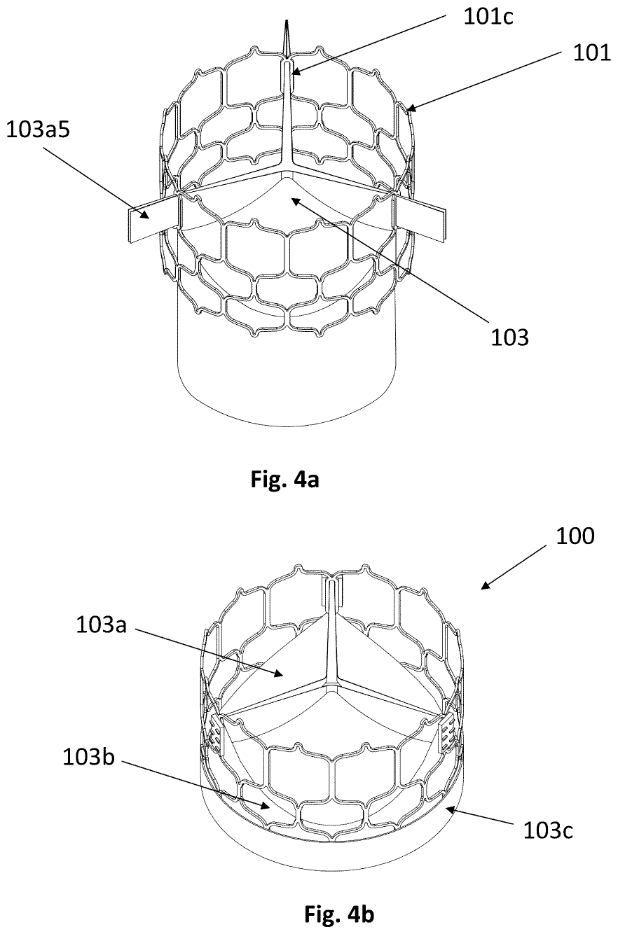

FIG. 4 depicts an exemplary method 400 to assemble of the prosthetic heart valve (for example, THV 100) with the polymeric structure 103 of the present invention. The method 400 begins at step 401 by attaching the valvular structure 103a to the frame 101 (as depicted in FIG. 4a).

The frame 101 may include a plurality of commissure windows 101c (as shown in FIG. 1) disposed at the outflow end 100b of the frame 101. The commissure windows 101c may be disposed equidistant from each other to receive the attachment portion 103a5 of the valvular structure 103a. In an exemplary embodiment, while the polymeric structure 103 is disposed within the frame 101, each pair of the attachment portion 103a5 of the valvular structure 103a are sutured to a respective commissure window 101c of the frame 101. The valvular structure 103a is sutured to the frame 101 such that the top portion 103a1 of the valvular structure 103a is disposed towards the outflow end 100b of the frame 101 and the bottom portion 103a2 of the valvular structure 103a is disposed towards the inflow end 100a of the frame 101.

In an alternate embodiment, the attachment portion 103a5 is fused to the respective commissure windows 101c of the frame 101. The attachment portion 103a5 may be fused to the commissure windows 101c with the help of a medical grade polymeric adhesives. The polymeric adhesives may include, without limitation acrylic-based adhesives, epoxy resins, polyurethanes, silicone-based adhesives, etc.

At step 403, the inner sealing member 103b may be attached to the frame 101 at least partially over an inner surface of the frame 101 towards the inflow end 100a. In an exemplary embodiment, the inner sealing member 103b is attached to the frame 101 by suturing the inner sealing member 103b using thin Polyethylene terephthalate (PET) sutures.

In an alternate embodiment, the inner sealing member 103b is attached to the frame 101 by fusing the inner sealing member 103b to the inner surface of the frame 101 with the help of a medical grade polymeric adhesives. The polymeric adhesives may include, without limitation acrylic-based adhesives, epoxy resins, polyurethanes, silicone-based adhesives, etc.

At step 405, the polymeric structure 103 may be folded inside-out at the fold ‘f’ such that the outer sealing member 103c is disposed at least partially over the outer surface of the frame 101 (depicted in FIG. 4b) towards the inflow end 100a.

Additionally or optionally, the outer sealing member 103c may be trimmed to reduce its height. The outer sealing member 103c may be trimmed by without limitation, sharp knife, razor blade, a scalpel, laser cutting, ultrasonic trimming, etc.

In an exemplary embodiment, the outer sealing member 103c is attached to the outside of the frame 101 by suturing the outer sealing member 103c to the frame 101 using thin Polyethylene terephthalate (PET) sutures.

In an alternate embodiment, the outer sealing member 103c is attached to the frame 101 by fusing the outer sealing member 103c to the outer surface of the frame 101 with the help of a medical grade polymeric adhesives. The polymeric adhesives may include, without limitation acrylic-based adhesives, epoxy resins, polyurethanes, silicone-based adhesives, etc.

Alternatively, the steps 401-405 may be skipped by preparing the polymeric structure 103 integrally with the frame 101. For example, after the mold 20 is subjected to the dipping cycles in step 203c of the method 200, the frame 101 is attached to the mold 20 such that the wings 20a1 of the mold 20 are received within and/or adjacent to respective commissure windows 101c of the frame 101. Thereafter, the mold 20 along with the frame 101 is further subjected to one or more dipping cycles. In an exemplary embodiment, the mold 20 is subjected to two dip cycles before the frame 101 is attached to the mold 20 to create a thin layer of the polymer solution over the mold 20. In an exemplary embodiment, the mold 20 along with the frame 101 is subjected to eight dip cycles to get the polymeric structure 103 on the frame 101 fused together. After the mold 20 along with the frame 101 is subjected to the one or more dip cycles, the above-described steps 203i-203m are followed to obtain the THV 100.

At step 407, the THV 100 may be crimped on a delivery catheter of a delivery device. Depending upon the material of the frame 101, the delivery device may be selected. For example, for a self-expandable THV 100, a self-expandable delivery device is selected. Similarly, for a balloon-expandable THV 100, a delivery device having a balloon catheter is selected. Further, in the case of a balloon-expandable THV 100, the THV 100 is not crimped on the delivery catheter. The balloon-expandable THV 100 is only crimped just before the THV 100 is implanted.

At step 409, the THV 100 is packed in a pre-defined box and subsequently subjected to sterilization. In an exemplary embodiment, the pre-defined box includes a Tyvek pouch. The packaged THV 100 may be sterilized by, without limitation gas sterilization and/or radiation sterilization. In an exemplary embodiment, the packaged THV 100 is sterilized using Ethylene oxide (EtO) gas. The sterilization of the packaged THV 100 preserves the properties of the polymeric structure 103. In an alternate embodiment, the packaged THV 100 is sterilized using gamma beams.

The invention will now be described in more detail by the following non-limiting examples:

### Example 1 (Prior Art): Preparation of a Polymeric Structure Using a Conventional Dip Coating Method

A polymer solution was prepared by dissolving 6 grams of polymer in 100 ml of THF solvent. The polymer was completely dissolved by stirring the polymer solution at 1500 rpm on a magnetic stirrer.

The mold 20 was dipped into the above prepared polymer solution such that the three quarters portion of the mold 20 were coated with the polymer solution. For each dip, the mold 20 was allowed to stay in the polymer solution for 3 seconds before withdrawing the mold 20 from the polymer solution. After withdrawing the mold 20 from the polymer solution, the mold 20 was maintained in an inverted position (i.e., the first portion 10a was held closed to the ground compared to the fourth portion 10d) such that excess polymer solution was allowed to run out for 10 seconds. The mold 20 was dipped for a total of 10 times.

After that, mold 20 was dried in a hot air oven a 60° C. for 3 hours to completely evaporate the solvent thereby creating the polymeric structure. Post drying, the polymeric structure was recovered from the mold 20 by carefully removing the polymeric structure using forceps. The resultant polymeric structure obtained was found to have bubbles and haziness with uneven thickness.

### Example 2 (Present Invention): Preparation of a Polymeric Structure 103 for a THV 100 from a Concentrated Polymer Solution

A polymer solution was prepared by dissolving 10 grams polymer in 100 ml of anhydrous THF solvent. The polymer was completely dissolved by stirring the polymer solution at 1500 rpm on a magnetic stirrer.

The mold 10 was dipped in the above prepared polymer solution such that the first portion 10a and most of the second section was coated with the polymer solution. The mold 10 was dipped only once and allowed to stay in the polymer solution for 10 seconds before the mold 10 was withdrawn from the polymer solution. Thereafter, the mold 10 was maintained in an inverted position (i.e., the first portion 10a was held closed to the ground compared to the fourth portion 10d) such that excess polymer solution was allowed to run out.

After that, the mold 10 was allowed to partially cure at 37° C. for 30 minutes inside a vacuum oven at a vacuum pressure of 650 mmHg. The partially cured mold 10 was then allowed to cure at 60° C. for 6 hours inside the vacuum oven at a vacuum pressure of 650 mmHg to completely evaporate the solvent thereby creating the polymeric structure 103. Post drying, the polymeric structure 103 was recovered from the mold 10 by carefully removing the polymeric structure 103 using forceps under running purified water.

The polymeric structure 103 was observed to have a thickness of about 180 microns. Further, the polymeric structure 103 was transparent without any bubbles and haziness.

### Example 3 (Present Invention): Preparation of the Polymeric Structure 103 of for the THV 100 from a Dilute Polymer Solution

A polymer solution was prepared by dissolving 6 grams of polymer in 90 ml anhydrous THF and 10 mL DMAc solvents. The polymer was completely dissolved by stirring the polymer solvent at 1500 rpm on a magnetic stirrer.

The mold 10 was dipped in the above prepared polymer solution such that the first portion 10a and most of the second section was coated with the polymer solution. For each dip, the mold 10 was allowed to stay in the polymer solution for 5 seconds before withdrawing the mold 10 from the polymer solution. After withdrawing the mold 10 from the polymer solution, the mold 10 was maintained in an inverted position (i.e., the first portion 10a was held closed to the ground compared to the fourth portion 10d) such that excess polymer solution was allowed to run out for 10 seconds. The mold 10 was dipped for a total of 9 times.

After that, the mold 10 was allowed to partially cure at 37° C. for 10 minutes inside a vacuum oven at a vacuum pressure of 650 mmHg. Thereafter, the partially cured mold 10 was then allowed to cure at 60° C. for 4 hours inside a vacuum oven at a vacuum pressure of 650 mmHg to completely evaporate the solvent thereby creating the polymeric structure 103. Post drying, the polymeric structure 103 was recovered from the mold 10 by carefully removing the polymeric structure 103 using forceps under running purified water.

The polymeric structure 103 was observed to have a thickness of about 180 microns. Further, the polymeric structure 103 was transparent without any bubbles and haziness.

### Example 4: In-Vitro Calcification Study

A calcification inducing solution was prepared by adding a calcium compound (for example, CaCl₂·2H₂O) and a phosphate (K₂HPO₄) salt in 0.05M tris buffer (pH 7.4). The resultant solution had a calcium to phosphate (Ca/PO₄) ratio of about 1.67.

A bovine pericardium tissue and the polymeric structure 103 of the present invention were cut into small pieces having length 3 cm and width 4 cm. The said pieces of the bovine pericardium tissue and the polymeric structure 103 were separately incubated at 37° C. in the above calcification inducing solution for 365 days inside shaking incubator.

After 365 days, it was observed that the bovine pericardium tissue showed signs of mild calcification. Whereas there was no visible sign of calcification on the polymeric structure 103 of the present invention. Thus, it was concluded that the polymeric structure 103 of the present invention is extremely resistant to calcification.

The aforesaid conclusion was further verified by placing the THV 100 having the polymeric structure 103 of the present invention inside an in vitro pulsatile tester machine for accelerated in vitro calcification study using the above calcification inducing solution. The pulsatile machine was operated at 5 Hz frequency. It was reaffirmed that the polymeric structure 103 of the THV 100 did not show any sign of calcification and continued to function properly in the accelerated working condition.

### Example 5: Surface Roughness Study

The surface roughness of the polymeric structure 103 of the present invention (having thickness 150 micron and 180 micron), conventional polymeric film and bovine pericardium tissue (in duplicate) were measured using Mitutoyo Surftest SJ-410 Surface Roughness Tester Machine and tabulated below for analysis. Generally, electro polished metal tubes such as Nitinol and Cobalt chromium are used for fabrication of cardiovascular stents that possess very smooth surface with no active site for binding of thrombus particles thereby resisting thrombogenesis. Thus, the surface roughness data of electro polished metal tubes were used as a control (reference) for this study.

TABLE 8
Surface Roughness Data

| | Roughness (Ra) | Average Roughness |
|---|---|---|
| Sample Name | value in μm | (Ra) value in μm |
| CoCr (cobalt | 0.139 | 0.130 |
| chromium) Tube-1 | | |
| CoCr (cobalt | 0.121 | |
| chromium) Tube-2 | | |
| Nitinol Tube-1 | 0.135 | 0.127 |
| Nitinol Tube-2 | 0.119 | |
| Bovine pericardium | 2.596 | 2.735 |
| tissue-1 | | |
| Bovine pericardium | 2.874 | |
| tissue-2 | | |
| Conventional | 1.329 | 1.191 |
| polymeric structure-1 | | |
| Conventional | 1.054 | |
| polymeric structure -2 | | |
| Polymeric structure - | 0.192 | 0.186 |
| present invention | | |
| (150 micron) | | |
| Polymeric structure - | 0.180 | |
| present invention | | |
| (180 micron) | | |

It should be noted that high Ra value corresponds to rough surface thereby being more susceptible to thrombogenesis. Thus, it was concluded that not only was the average roughness of the polymeric structure 103 less than the bovine pericardium tissues, the average roughness of the polymeric structure 103 was found to be relatively close to the average roughness of the electro polished metal tubes. Therefore, it was established that similar to electro polished metal tubes, the polymeric structure 103 of the present invention provides smooth surfaces which minimizes the propensity for platelet aggregation or any potential thrombus formation and/or pannus formation.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. A method to prepare a polymeric structure (103) for a prosthetic heart valve, the method comprising:
pre-processing a polymer for a pre-defined time to reduce residual moisture content of the polymer to less than 0.02% by weight of the polymer;
dissolving the pre-processed polymer in at least one solvent yielding a polymer solution, the polymer solution having a pre-defined concentration, the solvent having water content less than or equal to 0.005% (w/v);
subjecting a mold (10) to one or more dip cycles, wherein a dip cycle includes inserting the mold (10) in the polymer solution, maintaining the mold (10) inside the polymer solution for a first hold time period, and maintaining the mold (10) outside the polymer solution for a second hold time period;
subjecting the polymer solution on the mold (10) to a first curing technique at a first pre-defined temperature(s) and a first pre-defined pressure(s) for a first pre-defined time period(s);
subjecting the polymer solution on the mold (10) to a second curing technique at a second pre-defined temperature(s) and a second pre-defined pressure(s) for a second pre-defined time period(s) post step d to yield a polymeric structure (103), the polymeric structure (103) having a thickness ranging from 160 microns to 200 microns; and
peeling the polymeric structure (103) away from the mold (10).

2. The method as claimed in claim 1, wherein the step of pre-processing the polymer for the pre-defined time includes
selecting the polymer from a group of polyurethane or block co-polymers thereof including polyurethane, poly ether urethane, and polycarbonate urethane; and
drying the polymer by one of a vacuum desiccator, a vacuum oven, a desiccant type de-humidifying dryer, or a hot air oven for 12 hours to 24 hours.

3. The method as claimed in claim 1, wherein the step of dissolving the polymer in at least one solvent includes dissolving the polymer in at least one solvent at a concentration ranging from 3% to 11% (w/v).

4. The method as claimed in claim 1, wherein the step of dissolving the polymer in at least one solvent includes dissolving the polymer in at least one of dimethyl acetamide (DMAc), tetrahydrofuran (THF), or dimethyl sulfoxide (DMSO).

5. The method as claimed in claim 1, wherein the step of dissolving the polymer in at least one solvent includes dissolving the polymer in a blend of two solvents blended in a predefined ratio of 90:10, 80:20, 70:30, 60:40, 50:50, 40:60, 30:70, 20:80, or 10:90.

6. The method as claimed in claim 1, wherein the step of subjecting a mold (10) to the one or more dip cycles using the polymer solution includes partially inserting the mold (10) into the polymer solution up to a predefined height of the mold (10) based upon a height of an inner sealing member 103b and/or an outer sealing member 103c.

7. The method as claimed in claim 1, wherein the step of maintaining the mold (10) inside the polymer solution for the first hold time period includes maintaining the mold (10) within the polymer solution for 1 second to 10 second.

8. The method as claimed in claim 1, wherein the step of maintaining the mold (10) outside the polymer solution for a second hold time period includes maintaining the mold (10) out of the polymer solution for 1 second to 10 second.

9. The method as claimed in claim 1, wherein the step of subjecting the polymer solution to the first curing technique at the first pre-defined temperature(s) and the first pre-defined pressure(s) for the first pre-defined time period(s) includes placing the mold (10) inside one of hot air oven or vacuum oven at the first pre-defined temperature(s) ranging from 30° C. to 40° C. and the first pre-defined pressure(s) ranging from 650 mmHg to 700 mmHg for the first pre-defined time period(s) ranging from 5 minutes to 15 minutes.

10. The method as claimed in claim 1, wherein the step of subjecting the polymer solution to the second curing technique at the second pre-defined temperature(s) and the second pre-defined pressure(s) for the second pre-defined time period(s) includes placing the mold (10) inside one of hot air oven or vacuum oven at the second pre-defined temperature(s) ranging from 40° C. to 60° C. and the second pre-defined pressure(s) ranging from 650 mmHg to 700 mmHg for the second pre-defined time period(s) ranging from 3 hours to 4 hours.

11. The method as claimed in claim 1, wherein after preparing the polymeric structure (103), the method includes coating the polymeric structure (103) with a pre-defined coating.

12. The method as claimed in claim 1, wherein the step of subjecting a mold (10) to one or more dip cycles includes preparing the polymeric structure (103) integrally with the frame (101) by:
after subjecting the mold (20) to one or more dip cycles using the polymer solution, attaching a frame (101) to the mold (20), a wing(s) (20a1) of the mold (20) being received adjacent to respective commissure window(s) (101c) of the frame (101);
subjecting the mold (20) along with the frame (101) to one or more dip cycles.

13. A prosthetic heart valve, comprising:
a frame (101); and
a polymeric structure (103) attached to the frame (101), the polymeric structure (103) having a single partially continuous structure including three sections:
a valvular structure (103a) attached to an outflow end (100b) of the frame (101), the valvular structure (103a) disposed within the frame (101);
an inner sealing member (103b) attached to at least partially over an inner surface of the frame (101) towards an inflow end (100a) of the frame (101); and
an outer sealing member (103c) attached to at least partially over an outer surface of the frame (101) towards an inflow end (100a) of the frame (101);
wherein, the polymeric structure (103) is made from a polymer solution, the polymer solution including a polymer dissolved in at least one solvent, the polymer solution is subjected to a first curing technique at a first pre-defined temperature(s) and a first pre-defined pressure(s) for a first pre-defined time period(s);
wherein, the polymer solution is subjected to a second curing technique at a second pre-defined temperature(s) and second pre-defined pressure(s) for a second pre-defined time period(s);
wherein, the polymer is dehydrated; and
wherein, the at least one solvent is anhydrous.

14. The prosthetic heart valve as claimed in claim 13, wherein the valvular structure (103a) includes a single partially continuous structure having three pair of attachment portion (103a5) and three bottom edges (103a6).

15. The prosthetic heart valve as claimed in claim 13, wherein the polymeric structure (103) includes a thickness ranging from 160 to 200 microns.
